(19) United States
(12) Patent Application Publication
(10) Pub. No.: US 2012/0123491 A1
(43) Pub. Date: May 17, 2012

(54) DEFIBRILLATOR DEVICE WITH STATUS INDICATING TRANSPORT HANDLE

(76) Inventors: William Tom Hunter, Milwaukee, WI (US); Lawrence E. Murphy, Shorewood, WI (US)

(21) Appl. No.: 12/945,955

(22) Filed: Nov. 15, 2010

(51) Int. Cl. A61N 1/39

(52) U.S. Cl. 607/7; 607/5

(57) ABSTRACT

A device and method for automatically indicating an operating status of a defibrillator is provided. A defibrillator device includes a housing and a handle attached to the housing constructed at least partially of a light-transmitting material. A user interface is positioned within the housing that includes a plurality of controls thereon, with the plurality of controls enabling a user to control an operating status of the defibrillator device. A light source is positioned within the housing that is configured to illuminate the handle in one of a plurality of colors. The defibrillator device also includes a processor configured to determine the operating status of the defibrillator device and selectively control the light source to illuminate the handle in one of the plurality of colors based on the determined operating status of the defibrillator device.

### BACKGROUND OF THE INVENTION

Embodiments of the invention relate generally to a defibrillator and, more particularly, to a defibrillator that provides for automated generation of an alert or notification that the defibrillator is in a fully charged state and ready to deliver an electric shock to a patient.

Sudden cardiac arrest is a leading cause of death in the developed world. Most episodes of sudden cardiac arrest are caused by ventricular fibrillation, in which the muscle fibers of the heart, which normally contract in a coordinated manner to pump blood around the arterial system of the body, contract instead in an uncoordinated manner. This interrupts what should be the normal flow of blood to the body. The heart, once in ventricular fibrillation, can be forced back into a normal heart rhythm by the application of an electric shock. The application of this shock, known as defibrillation, clears the heart of the abnormal electrical activity by depolarizing a critical mass of myocardial cells to allow normal spontaneous organized myocardial depolarization to resume. Studies have shown that, in order for defibrillation to be effective, the defibrillation shock must be delivered to the patient within minutes of the onset of ventricular fibrillation. The commonly used means to apply this electrical defibrillation shock is a defibrillator. In operation, a defibrillator applies the electric shock through two electrodes placed on the chest of the victim.

While the shock generated by the defibrillator has been proven as an effective way for forcing the heart back into a normal heart rhythm, it is recognized that such a shock can pose a hazard to others in the vicinity that are in contact with the patient receiving the shock. In operation of a defibrillator, a healthcare professional will typically power ON the device and select an energy level. The healthcare professional will then press a Charge key, wait for the device to indicate that it has reached the selected energy, and then press a “Shock” key to deliver the electrical energy to the patient through electrodes pads or paddle, after verbally announcing “clear” to indicate to others to stand clear.

However, while the status of the device indicating that it has reached the selected energy is visible to the healthcare professional, it may not necessarily be visible to other medical personnel in the area. As such, absent any indication by the operator to stand clear from the patient in preparation for the shock to be applied, other medical personnel in the area may have no other means by which to ascertain that the defibrillator has reached the selected energy and is ready to deliver the electric shock. This failure to notify other medical personnel in the area that the defibrillator is ready to deliver the electric shock may lead to a situation where such medical personnel are still in contact with the patient when the operator applies the electric shock to the patient, which is a highly undesirable result.

Therefore, it would be desirable to design a defibrillator that provides an alert or notification to medical personnel indicating that the defibrillator is ready to deliver an electric shock to a patient. It would also be desirable for the defibrillator to provide such an alert or notification in an automated fashion so as to eliminate the possibility of operator error in generating the alert.

### BRIEF DESCRIPTION OF THE INVENTION

Embodiments of the invention are directed to a system and method that provides for automated indication of an operating status of the defibrillator. A defibrillator includes a handle that is selectively caused to illuminate based on the operating status of the defibrillator device. A multi-colored light source is positioned within the handle and is selectively controlled to illuminate the handle in one of a plurality of colors specific to an operating status of the defibrillator device.

In accordance with one aspect of the invention, a defibrillator device includes a housing, a handle attached to the housing and being constructed at least partially of a light-transmitting material, a user interface positioned within the housing and including a plurality of controls thereon, wherein the plurality of controls enable a user to control an operating status of the defibrillator device, and a light source positioned within the housing and being configured to illuminate the handle in one of a plurality of colors. The defibrillator device also includes a processor configured to determine the operating status of the defibrillator device and selectively control the light source to illuminate the handle in one of the plurality of colors based on the determined operating status of the defibrillator device.

In accordance with another aspect of the invention, a defibrillator device includes a housing, a handle attached to the housing and being constructed at least partially of a light-transmitting material, and a user interface provided on a front surface of the housing to control an operational mode of the defibrillator device. The defibrillator device also includes an electrically activatable multi-colored light source positioned within the handle and configured to illuminate the handle in one of a plurality of colors in response to an activation signal and a processor operationally coupled to the multi-colored light source, with the processor being programmed to determine a current operational mode of the defibrillator device and issue an activation signal to the multi-colored light source to selectively illuminate the handle in one of the plurality of colors based on the determined operational mode of the defibrillator device.

In accordance with yet another aspect of the invention, a method for indicating a current operating status of a defibrillator device includes the step of determining an operational mode of a defibrillator device that includes an illuminatable handle constructed at least partially of a light-transmitting material, wherein the operational mode comprises one of an energy selection mode, a charge initiation mode, and a fully charged mode. The method also includes the step of selectively illuminating the handle of the defibrillator device based on the determined operational mode of the defibrillator device, wherein the step of selectively illuminating the handle further includes activating a first set of light emitting diodes (LEDs) positioned within the handle and having a first color upon a determination that the defibrillator device is in the energy selection mode so as to illuminate the handle in the first color upon activation of the first set of LEDs, activating a second set of LEDs positioned within the handle and having a second color upon a determination that the defibrillator device is in the charge initiation mode so as to illuminate the handle in the second color upon activation of the second set of LEDs, and activating a third set of LEDs positioned within the handle and having a third color upon a determination that the defibrillator device is in the fully charged mode so as to illuminate the handle in the third color upon activation of the third set of LEDs.

Various other features and advantages will be made apparent from the following detailed description and the drawings.

### BRIEF DESCRIPTION OF THE DRAWINGS

The drawings illustrate preferred embodiments presently contemplated for carrying out the invention.

In the drawings:

FIG. 1 is a pictorial view of a defibrillator according to an embodiment of the invention.

FIG. 2 is a block schematic diagram of the defibrillator of FIG. 1 according to an embodiment of the invention.

FIG. 3 is a flowchart illustrating a technique for indicating a current operating status of a defibrillator according to an embodiment of the invention.

### DETAILED DESCRIPTION

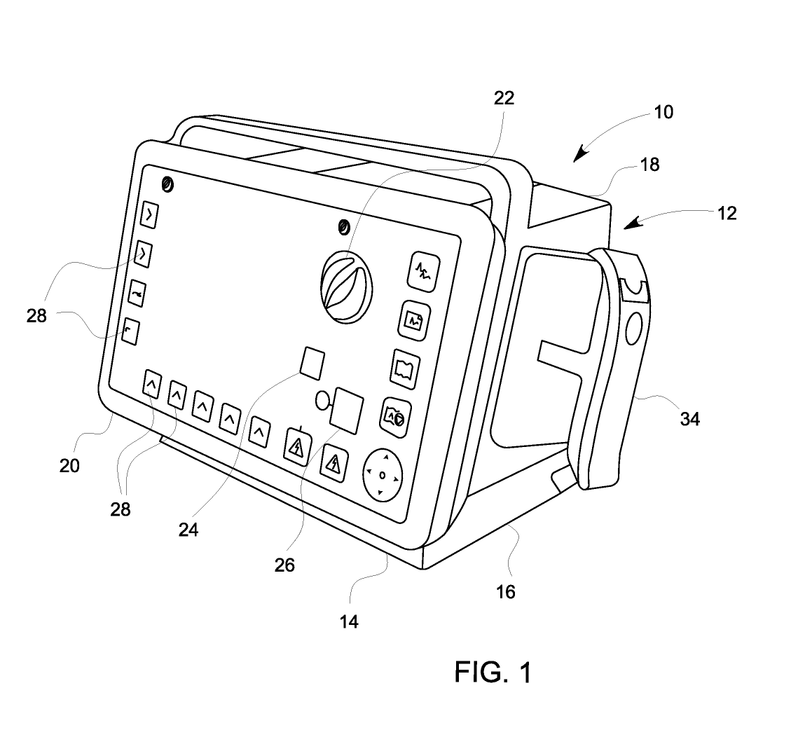

Referring to FIG. 1, a portable defibrillator 10 is shown according to an embodiment of the invention. The defibrillator 10 includes defibrillator and monitor circuitry (not shown) and a body 12 for housing the defibrillator and monitor circuitry. The body 12 has a front 14, a base 16, and a rear 18. Progressing upwards from the base 16, the front 14 is angled back toward the rear 18.

Disposed on the front 14 is a user interface panel 20 for exchanging information with an operator of the defibrillator 10. According to the embodiment of defibrillator 10 illustrated in FIG. 1, the user interface panel 20 includes an energy selection knob or dial 22 by way of which the operator adjusts and sets an energy level at which an electrical shock will be applied to the patient. A charge initiation button 24 and a charge delivery button 26 are also included on user interface panel 20, with charge initiation button 24 being provided for the operator to begin a charging process of defibrillator 10 and charge delivery button 26 being provided for the operator to apply the electric charge to the patient. The user interface panel 20 may also include a variety of additional knobs, buttons, keys, or other input means 28 for receiving information from the user.

Preferably, the user interface panel 20 also has a screen 30 for displaying information to the user. The user interface panel 20, including the screen 30, is disposed on the front 14 of the body 12 to face at an upward angle relative to the base 16 of the defibrillator. In a preferred embodiment of the invention, the defibrillator body 12 is shaped to secure the user interface panel 20 at an upward angle, thereby providing medical personnel standing above the defibrillator with an enhanced ability to view the screen and access the defibrillator controls.

The defibrillator 10 shown in FIG. 1 also includes electrodes or shock paddles 32 and leads (not shown) for applying a defibrillating pulse to a patient. The leads electrically couple the electrodes 32 with the defibrillator circuitry, and conduct defibrillating pulses. The defibrillator circuitry, electrodes, and leads, and the interconnections thereof, are of conventional design and are constructed by means known in the art.

As further shown in FIG. 1, defibrillator 10 also includes a handle 34 extending upward from body 12 adjacent to face 14. According to embodiments of the invention, handle 34 is illuminated in different colors during operation of defibrillator 10 to indicate an operating status of the defibrillator. That is, handle 34 is illuminated in a first color when the device is powered ON, indicating a safe condition. The handle 34 is then illuminated in a second color, indicating that defibrillator 10 is ready to begin charging and/or is currently charging. When defibrillator 10 is fully charged, the handle 34 is illuminated in a third color, indicating that the defibrillator 10 is ready to apply an electric charge to the patient. To enable such changes in color indicative of different operating states, handle 34 is constructed of a light-transmitting material, such as a crystal-clear polycarbonate material, that functions to “pipe” light through the handle, although it is recognized that other transparent or translucent materials could also be used to form handle 34. The color of handle 34 can thus be controlled based on a color of the light piped therethrough.

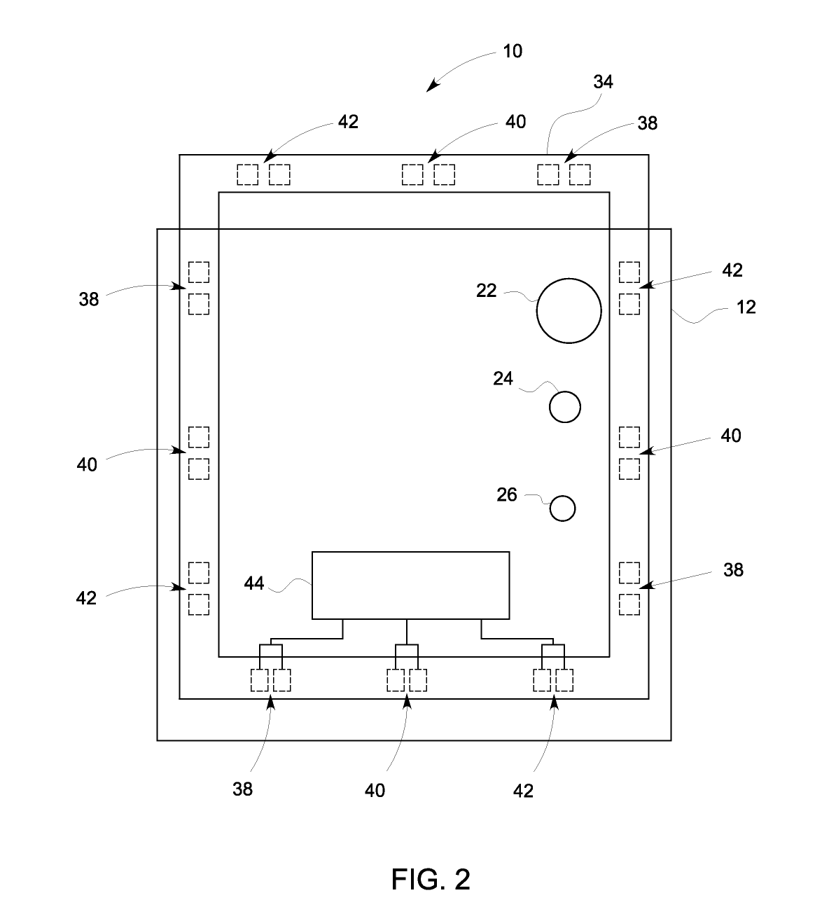

According to one embodiment of the invention, handle 34 is caused to be illuminated in one of several colors by way of a multi-colored light source included in defibrillator 10. Referring now to FIG. 2, the multi-colored light source 36 is shown positioned within the handle 34 of the defibrillator. According to one embodiment of the invention, multi-colored light source 36 is formed from a plurality of light-emitting diodes (LEDs), although other types of light sources are also envisioned (e.g. filament light bulbs). As shown in the embodiment of FIG. 2, the plurality of LEDs includes a first set of LEDs 38 in a first color, a second set of LEDs 40 in a second color, and a third set of LEDs 42 in a third color, so as to control a color in which handle is illuminated. LEDs can be provided in blue, yellow, and red colors, for example, to indicate distinct operating statuses of defibrillator 10. While multi-colored light source 36 is shown and described as including three sets of LEDs 38, 40, 42, it is recognized that a greater or lesser number of sets of LEDs could be provided, such that handle 34 could be illuminated in a greater or lesser number of colors, such as two distinct colors or four distinct colors, for example.

As shown in FIG. 2, according to one embodiment of the invention, the first, second, and third sets of LEDs 38, 40, 42 of light source 36 are positioned within a section of handle 34 enclosed within the body 12. In such an embodiment, handle 34 functions to “pipe” light from the illuminated set of LEDs 38, 40, 42 through the remainder of the handle 34, such that the entirety of handle 34 is illuminated in a determined/specified color. Alternatively, and as shown in phantom in FIG. 2 individual LEDs can be evenly spaced throughout the handle 34. Thus, individual LEDs forming each of the first set of LEDs 38, the second set of LEDs 40, and the third set of LEDs 42, can be positioned throughout the handle 34. In either embodiment, the entirety of handle 34 is illuminated in a determined/specified color, based on the selective activation of one of the first, second, and third sets of LEDs 38, 40, 42.

To provide ease of use to an operator, it is recognized that the illumination of handle 34 in differing colors can be tied to a color scheme of controls provided on user interface panel 20. More specifically, the illumination of handle 34 can be correlated with color coded controls of user interface panel 20, including selection dial 22, charge initiation button 24, and charge delivery button 26. According to an exemplary embodiment of the invention, the selection dial 22 and the first set of LEDs 38 are provided in blue, the charge initiation button 24 and the second set of LEDs 40 are each provided in yellow, and the charge delivery button 26 and the third set of LEDs 42 are each provided in red.

In operation, the first, second, and third sets of LEDs 38, 40, 42 are selectively caused to illuminate by a processor 44 based on a sensed operating status/condition of defibrillator 10. That is, processor 44 is configured to monitor a status of defibrillator 10 and, based on a determined status, cause one of the first, second, and third sets of LEDs 38, 40, 42 to illuminate according to the determined status. According to an embodiment of the invention, processor 44 is able to detect actuation of selection dial 22, charge initiation button 24, and charge delivery button 26 to determine an operating status of defibrillator 10. Additionally, processor 44 is able to sense a present level of charge of the defibrillator 10, so as to provide for a detection of when defibrillator 10 is in a partially or fully charged state. Based on the determination of a current operating status/condition of defibrillator 10 by processor 44, the processor 44 generates an activation signal that is issued/transmitted to one of the first, second, and third sets of LEDs 38, 40, 42. The activation signal causes a specified set of LEDs 38, 40, 42 to activate (i.e., illuminate), thereby also causing the handle 34 to become illuminated in a color corresponding to the determined operational mode of the defibrillator 10.

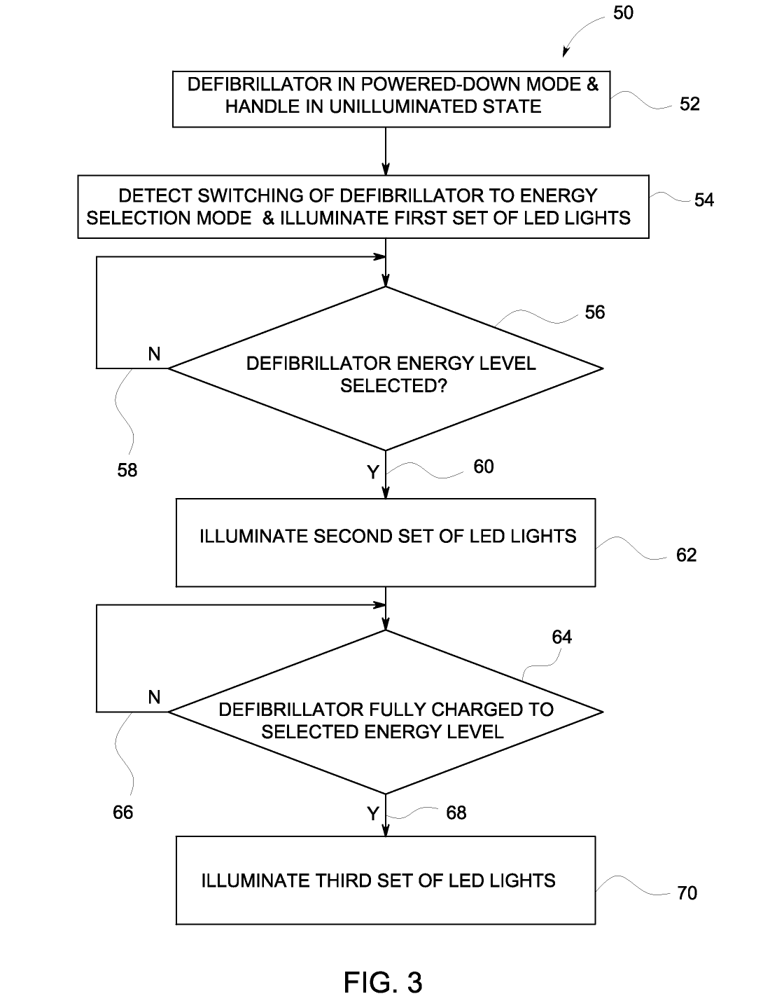

Referring now to FIG. 3, and with reference being made to FIGS. 1 and 2, a technique 50 is provided for indicating a current operating status of a defibrillator 10. According to technique 50, the handle 34 of defibrillator 10 is initially in an unilluminated state when defibrillator 10 is in a powered-down or “OFF” condition, as indicated at STEP 52. Upon a switching of defibrillator 10 to a powered-up or “ON” condition, processor 44 determines the defibrillator 10 to be in an energy selection mode and causes first set of LED lights 38 to become illuminated at STEP 54, such that handle 34 is illuminated in a first color. For example, a set of blue LED lights 38 can be activated by processor 44 (via an activation signal) to be become illuminated, such that handle 34 is illuminated in blue. Illumination of handle 34 in blue thus indicates to an operator, and other healthcare personnel in the area, that defibrillator 10 is powered ON and is in a “safe” condition. Illumination of handle 34 in blue also prompts the operator to select an energy level to which the defibrillator 10 is to be charged.

In a next step of technique 50, the defibrillator 10 is monitored to detect when an energy level has been selected by the operator, with a determination being made at STEP 56 as to whether an energy level has been selected. For example, a determination can be made at STEP 56 as to whether selection dial 22 has been actuated by the operator to select a specific energy level to which defibrillator 10 will be charged. If no energy level has been selected by the operator 58, technique 50 then continues to monitor defibrillator 10 for selection of an energy level. Upon a determination/detection that an energy level has been selected by the operator 60, such as by detecting an actuation of selection dial 22, technique 50 then continues with processor 44 causing second set of LED lights 40 to become illuminated at STEP 62, such that handle 34 is illuminated in a second color. For example, a set of yellow LED lights 40 can be activated by processor 44 (via an activation signal) to be become illuminated, such that handle 34 is illuminated in yellow. Illumination of handle 34 in yellow thus prompts the operator to press charge initiation button 24 so as to begin charging of defibrillator 10 toward the selected energy level. Illumination of handle 34 in yellow also alerts other healthcare personnel in the area that defibrillator 10 is ready to initiate charging and/or is in the process of charging, thereby indicating a “caution” condition.

Upon initiation of a charging of defibrillator 10, technique 50 continues by monitoring a charge level of defibrillator 10, with a determination being made at STEP 64 as to whether the defibrillator 10 is fully charged to the selected energy level. If it is determined that defibrillator 10 is not yet fully charged 66, technique 50 then continues to monitor the charge level of the defibrillator 10. Upon a determination/detection that defibrillator 10 is fully charged to the desired energy level 68, technique 50 then continues with processor 44 causing a third set of LED lights 42 to become illuminated at STEP 70, such that handle 34 is illuminated in a third color. For example, a set of red LED lights 42 can be activated by processor 44 (via an activation signal) to be become illuminated, such that handle 34 is illuminated in red. Illumination of handle 34 in red thus indicates to an operator, and other healthcare personnel in the area, that defibrillator 10 is in a fully charged state and ready to apply a shock to a patient. The illumination of handle 34 in red thus provides notice to other healthcare personnel in the area of a “danger” condition that requires all personnel to stand clear of the defibrillator 10 and the patient.

Defibrillator 10 thus is configured to indicate to an operator and surrounding healthcare personnel a current operating status (i.e., operational mode) of the defibrillator 10 based on a selective color-controlled illumination of handle 34. By selectively controlling illumination of handle 34 of defibrillator 10 in one of several designated colors, an alert or notification is provided to medical personnel indicative of a specific state of operation of the defibrillator. Thus, for example, an alert can be provided to medical personnel indicating that the defibrillator 10 is ready to deliver an electric shock to a patient, so as to provide an additional safeguard to those medical personnel. The defibrillator 10 provides such an alert or notification in an automated fashion, thereby also eliminating the possibility of operator error in generating the alert.

Therefore, according to one embodiment of the invention, a defibrillator device includes a housing, a handle attached to the housing and being constructed at least partially of a light-transmitting material, a user interface positioned within the housing and including a plurality of controls thereon, wherein the plurality of controls enable a user to control an operating status of the defibrillator device, and a light source positioned within the housing and being configured to illuminate the handle in one of a plurality of colors. The defibrillator device also includes a processor configured to determine the operating status of the defibrillator device and selectively control the light source to illuminate the handle in one of the plurality of colors based on the determined operating status of the defibrillator device.

According to another embodiment of the invention, a defibrillator device includes a housing, a handle attached to the housing and being constructed at least partially of a light-transmitting material, and a user interface provided on a front surface of the housing to control an operational mode of the defibrillator device. The defibrillator device also includes an electrically activatable multi-colored light source positioned within the handle and configured to illuminate the handle in one of a plurality of colors in response to an activation signal and a processor operationally coupled to the multi-colored light source, with the processor being programmed to determine a current operational mode of the defibrillator device and issue an activation signal to the multi-colored light source to selectively illuminate the handle in one of the plurality of colors based on the determined operational mode of the defibrillator device.

According to yet another embodiment of the invention, a method for indicating a current operating status of a defibrillator device includes the step of determining an operational mode of a defibrillator device that includes an illuminatable handle constructed at least partially of a light-transmitting material, wherein the operational mode comprises one of an energy selection mode, a charge initiation mode, and a fully charged mode. The method also includes the step of selectively illuminating the handle of the defibrillator device based on the determined operational mode of the defibrillator device, wherein the step of selectively illuminating the handle further includes activating a first set of light emitting diodes (LEDs) positioned within the handle and having a first color upon a determination that the defibrillator device is in the energy selection mode so as to illuminate the handle in the first color upon activation of the first set of LEDs, activating a second set of LEDs positioned within the handle and having a second color upon a determination that the defibrillator device is in the charge initiation mode so as to illuminate the handle in the second color upon activation of the second set of LEDs, and activating a third set of LEDs positioned within the handle and having a third color upon a determination that the defibrillator device is in the fully charged mode so as to illuminate the handle in the third color upon activation of the third set of LEDs.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A defibrillator device comprising:
a housing;
a handle attached to the housing and being constructed at least partially of a light-transmitting material;
a user interface positioned within the housing and including a plurality of controls thereon, wherein the plurality of controls enable a user to control an operating status of the defibrillator device;
a light source positioned within the housing and being configured to illuminate the handle in one of a plurality of colors; and
a processor configured to:
determine the operating status of the defibrillator device; and
selectively control the light source to illuminate the handle in one of the plurality of colors based on the determined operating status of the defibrillator device.

2. The defibrillator device of claim 1 wherein the plurality of controls of the user interface comprises:
an energy level selection control to provide for user selection of an energy level at which a shock is applied to a subject;
a charge initiation control to provide for user initiation of charging of the defibrillator device; and
a charge delivery control to provide for application of a shock to the subject at the selected energy level.

3. The defibrillator device of claim 2 wherein the light source comprises:
a first set of light-emitting diodes (LEDs) configured to illuminate in a first color;
a second set of LEDs configured to illuminate in a second color; and
a third set of LEDs configured to illuminate in a third color.

4. The defibrillator device of claim 3 wherein the processor is configured to cause the first set of LEDs to illuminate upon a powering-on of the defibrillator device.

5. The defibrillator device of claim 3 wherein the processor is configured to cause the second set of LEDs to illuminate upon operator actuation of the energy level selection control.

6. The defibrillator device of claim 3 wherein the processor is configured to cause the third set of LEDs to illuminate when the charge level reaches the selected energy level.

7. The defibrillator device of claim 3 wherein the plurality of controls of the user interface is configured such that:
the energy level selection control is colored in the first color to correlate with the first set of LEDs;
the charge initiation control is colored in the second color to correlate with the second set of LEDs; and
the charge delivery control is colored in the third color to correlate with the third set of LEDs.

8. The defibrillator device of claim 1 wherein the processor is configured to determine the operating status of the defibrillator device based on a user actuation of the user interface.

9. The defibrillator device of claim 1 wherein the processor is configured to determine the operating status of the defibrillator device based on a detected charge level of the defibrillator device.

10. The defibrillator device of claim 1 wherein the handle is constructed of a clear polycarbonate material configured to pipe light emitted from the light source through the handle, so as to illuminate the entirety of the handle in one of the plurality of colors.

11. The defibrillator device of claim 1 wherein the light source is positioned within the handle.

12. A defibrillator device comprising:
a housing;
a handle attached to the housing and being constructed at least partially of a light-transmitting material;
a user interface provided on a front surface of the housing to control an operational mode of the defibrillator device;
an electrically activatable multi-colored light source positioned within the handle and configured to illuminate the handle in one of a plurality of colors in response to an activation signal; and
a processor operationally coupled to the multi-colored light source, the processor being programmed to:
determine a current operational mode of the defibrillator device; and
issue an activation signal to the multi-colored light source to selectively illuminate the handle in one of the plurality of colors based on the determined operational mode of the defibrillator device.

13. The defibrillator device of claim 12 wherein the processor is further programmed to:
activate a first set of LEDs in the multi-colored light source upon detection of a first operational mode of the defibrillator device, such that the handle is illuminated in a first color;
activate a second set of LEDs in the multi-colored light source upon detection of a second operational mode of the defibrillator device, such that the handle is illuminated in a second color; and
activate a third set of LEDs in the multi-colored light source upon detection of a third operational mode of the defibrillator device, such that the handle is illuminated in a third color.

14. The defibrillator device of claim 13 wherein the first operational mode comprises an uncharged state, the second operational mode comprises a partially charged state, and the third operational mode comprises a fully charged state.

15. The defibrillator device of claim 13 wherein the second operational mode is initiated upon user selection of a desired energy level at which to shock a subject.

16. The defibrillator device of claim 12 wherein the user interface comprises:
an energy level selection control to provide for user selection of a desired energy level at which a shock is applied to a subject;
a charge initiation control to provide for user initiation of charging of the defibrillator device; and
a charge delivery control to provide for application of a shock to the subject at the selected energy level.

17. The defibrillator device of claim 12 wherein the handle is constructed of a clear polycarbonate material configured to pipe light emitted from the light source through the handle, so as to illuminate the entirety of the handle in one of the plurality of colors.

18. A method for indicating a current operating status of a defibrillator device, the method comprising:
determining an operational mode of a defibrillator device that includes an illuminatable handle constructed at least partially of a light-transmitting material, wherein the operational mode comprises one of an energy selection mode, a charge initiation mode, and a fully charged mode; and
selectively illuminating the handle of the defibrillator device based on the determined operational mode of the defibrillator device, wherein selectively illuminating the handle comprises:
activating a first set of light emitting diodes (LEDs) having a first color upon a determination that the defibrillator device is in the energy selection mode, the first set of LEDs being positioned within the handle such that the handle is illuminated in the first color upon activation of the first set of LEDs;
activating a second set of LEDs having a second color upon a determination that the defibrillator device is in the charge initiation mode, the second set of LEDs being positioned within the handle such that the handle is illuminated in the second color upon activation of the second set of LEDs; and
activating a third set of LEDs having a third color upon a determination that the defibrillator device is in the fully charged mode, the third set of LEDs being positioned within the handle such that the handle is illuminated in the third color upon activation of the third set of LEDs.

19. The method of claim 18 wherein activating one of the first set of LEDs, the second set of LEDs, and the third set of LEDs comprises selectively issuing an activation signal from a processor to one of the first set of LEDs, the second set of LEDs, and the third set of LEDs based on the determined operational mode of the defibrillator device.

20. The method of claim 18 wherein determining the operational mode of the defibrillator device comprises at least one of:
determining the operational mode based on operator interaction with a user interface of the defibrillator device; and
determining the operational mode based on a detected charge level of the defibrillator device.
